(19) United States
(12) Patent Application Publication
(10) Pub. No.: US 2010/0098794 A1
(43) Pub. Date: Apr. 22, 2010

(54) Topical anti-wrinkle and anti-aging moisturizing cream

(76) Inventor: Gérard Armand, Hastings-on-Hudson, NY (US)

Correspondence Address: Gerard Armand, 244 Mt. Hope Blvd, Hastings-on-Hudson, NY, 10706, US

(21) Appl. No.: 12/589,088

(22) Filed: Oct. 19, 2009

Related U.S. Application Data
(60) Provisional application No. 61/106,413, filed on Oct. 17, 2008.

(51) Int. Cl. A61K 36/185; A61K 31/728; C12N 9/26; A61P 17/02

(52) U.S. Cl. 424/776; 514/54; 435/201

(57) ABSTRACT

The present invention relates to a topical anti-wrinkle and anti-aging moisturizing cream designed (formulated) to repair and restore human adult skin to its original youthful state. This has successfully been achieved by using a water based molecular mixture of high molecular weight (0.5 to 2.0 million Daltons) hyaluronic acid (HA) and of low molecular weight hyaluronic acid oligosaccharides (small HA fragments less than 12000 Daltons molecular weight) to create this unique anti-aging moisturizer. An equal mixture of high molecular HA and HA-oligosaccharides at concentrations of 0.1-0.2% was found to be essential for providing our moisturizer its unique ability to repair and rejuvenate adult human skin.

This application claims the benefit of U.S. Provisional Application No. 61/106,413, filed Oct. 17, 2008, which is incorporated here in its entirety.

### FIELD OF INVENTION

This invention relates to an aqueous composition of hyaluronic acid (HA) also referred as hyaluronan comprising equal amounts (w/w) of high molecular weight HA (0.5 to 2 million Daltons) and of low molecular weight saturated HA-oligosaccharides (500 to 12,000 Daltons) for creating a cosmetic formulation with benefits of removing wrinkles and slowing down the aging process of human skin.

### BACKGROUND

### Prior Art

Hyaluronic acid (HA), referred also as hyaluronan as well as its salts, such as sodium hyaluronate, potassium hyaluronate, etc., is ubiquitous in all connective tissue. It is one of the most important and abundant polysaccharides in the animal kingdom. It is also present in some bacterial cell walls (Meyer et al., BBA. 21, 506, 1956; Balazs et al., Arch. Biochem. Biophys. 81, 464, 1959). Unlike the other animal polysaccharides which appear in nature as proteogloycans, i.e. covalently bound to proteins, hyaluronic acid appears in its native state as glycosaminoglycan, not covalently bound to any protein core. Hyaluronic acid was first isolated from bovine vitreous in 1934 by Meyer and Palmer (Meyer, K. & Palmer, J. W., J. Biol. Chem. 107, 629, 1934) and, in subsequent years, from a variety of different connective tissues including skin (Meyer, K. & Chaffee, E., JBC 133, 491, 1941).

Meyer and his associates also characterized the primary structure of hyaluronic acid (HA) by chemically isolating the basic crystalline disaccharide unit, hyalobiuronic acid from both acid hydrolysates and enzymatic digests of hyaluronic acid. The structure of this disaccharide was shown to be beta-1, 3-D-glucuronido-N-acetyl-D-glucosamine (Weissmann, B. & Meyer, K., J. Amer. Chem. Soc. 76, 1753-1757, 1954). Purified preparations of hyaluronic acid appeared to be very polydispersed, but possessed as yet no proven chemical heterogeneity. The polydispersity of HA has been studied in great detail by various investigators, including myself, using a variety of different techniques. (Laurent, T. C., J. BioChem. 216, 263, 1955; Laurent et al., Biochim. Acta 42, 476, 1960; Nichol, L. W. et al. Biochem. J. 102, 407, 1967; Bettelheim, F. A. and Balazs, E. A. Biochim. Biophys. Acta 158, 309, 1968; Armand, G. and Reyes, M. Biochem. Biophys. Res. Comm. 112, 175, 1983; Armand, G. and Chakrabarti, B. Curr. Eye Res. 6, 445, 1987). The molecular weight of purified hyaluronic acid (HA) preparations varies dramatically from 100,000 to several million Daltons, depending on the source of the material as well as the methods used for their extraction and purification (Balazs, E. A. U.S. Pat. No. 4,303,676)

In general, most connective tissue polysaccharides represent families of different macromolecules with precise structures and definite biological functions. For example, chondroitin-4 and 6-sulfate differ only in the position of the sulfate group, yet they appear to be chemically and physiologically two distinct entities. In contrast, no such behavior has yet been observed with hyaluronic acid. The general observation is that HA is polydispersed, although no biological significance has been found to be associated with this apparent polydispersity.

In mammalian connective tissue, multiple biological roles have been ascribed to hyaluronic acid (HA). For example, in the synovial cavity, as in other joints, HA has been postulated to act as a lubricating agent and a shock absorber thereby protecting surfaces of cartilage from mechanical damage. Again, in eye tissue the biological role of HA seems to be even more diverse from stabilizing the structure of the gel vitreous by binding to the collagen fibers to regulating the intra-ocular pressure of the eye by its presence in the trabecular meshwork (Barany, E. H. & Scotchbrook, S., Acta Phys. Scand. 30, 340, 1954; Schachtschabel, D. O. et al., Exp. Eye Res. 24, 71, 1977). With no less interest, hyaluronic acid has been shown to control a variety of biological events occurring at the cellular level, i.e., stimulating the function of polymorphonuclear leukocytes, both in vitro and in vivo, the aggregation of cartilage cells and the phagocytosis of macrophages, as well as the inhibition of leukocyte chemotaxis and cell infectivity, etc. (Brandt, K. D., Arthritis and Rheum, 1, 3, 308, 1970; Clarris, B. J., Ann. Rheum. Dis. 33, 240, 1974; Rydell, N. W., Acta, Orthop. Scand. 41, 307, 1970; Forester, J. V. & Balazs, E. A., Immunology 40, 435, 1970; Hakanson, L. et al., J. Clin. Invest. 66, 298, 1980).

Commercial purified hyaluronic acid isolated both from bacteria and rooster combs are currently being used for the therapy of different medical indications, i.e. as vicoelastic materials for eye surgery (U.S. Pat. No. 4,141,973) as well as for the treatment of arthritis (U.S. Pat. No. 7,456,275) and emphysema (Cantor, J. U.S. Pat. No. 5,633,003. In the skin, as in other mammalian tissues, the biological role of hyaluronic acid is paramount. Its presence in skin tissues was first signaled from chemical and histological studies carried out by Van Lier in 1908, later by Claude in 1940, and finally firmly established by Meyer and Chaffee in 1941 (Van Lier, E. H. B, Z. Physiol. Chem. 6, 177, 1909; Claude, A. Proc. Exp. Biol. and Med. 43, 487, 1940; Meyer, K. and Chaffee, E. J. Biol. Chem. 138, 491, 1941).

In the human skin, similar to many other animals, the sulfated polysaccharides such as chondroitin 6-sulfate, keratan sulfate, dermatan sulfate, heparatin sulfate, etc., are also present. It is worthy to note that during the pre-natal and post-natal development of human skin, the concentration of hyaluronic acid in that tissue is very high. Interestingly, while the concentration of HA diminishes during the maturation and aging of the skin, the concentration of sulfated polysaccharides, especially dermatan sulfate, increases. In skin, as well as in other connective tissue, dermatan sulfate has been shown to be associated with polymeric or cross-linked collagen and elastin. Such cross-links are primarily responsible for the appearance of deep folds or wrinkles on the skin. Clinically, no other organ of the human body reflects the aging process as clearly as does the skin. Throughout the ages scientists and cosmetic chemists have sought to create cosmetic formulations that could not only repair skin damage caused by external agents such as burns, extreme dryness, etc., but also duplicate the early conditions of skin development. They have met with minimal success.

In 1981, Balazs introduced hyaluronic acid as an excellent moisturizing agent to be used in cosmetic formulations (Balazs, E. A., U.S. Pat. No. 4,303,676). In 1982, Estee Lauder marketed Night Repair, a product containing high molecular weight hyaluronic acid. Today, because of its water-binding and transferring properties, the use of high molecular weight hyaluronic acid (HA) as a prime moisturizing agent in skin care products has become common place. However, there are some drawbacks associated with the use of high molecular weight HA in topical cosmetic formulations as they do not penetrate the skin or have very limited capacity to do so. On the other hand, recent reports in the scientific literature pertaining to in vitro and in vivo studies evaluating the effects of lower molecular weight HA (50,000-800,000 Daltons) on human skin have shown significant benefits in restoring skin elasticity and firmness and (Guillaumie, F. et al. Cosmetics and Toiletries, 121, #4, 51, 2006: Malle, B. M. et al., Abstr. #43, p. 72, The 7^{th} Internat. Conference on Hyaluronan, Charleston, S.C., USA, Apr. 22-27, 2007; Abdellaoui, et al. US Pat. Appl. No 20080274999; Abdellaoui, et al. US Pat. Appl. No 20080003271). In spite of their merits, all these studies were carried out using low molecular weight hyaluronic acid fractions of larger molecular volume than the oligo-saccharides used in the present invention.

The approach taken in the present invention is unique and totally different from anything used in previous cosmetic products. Here, an aqueous mixture of high molecular weight HA and HA-oligosaccharides (500-12,000 Daltons) is used to create the anti-aging cream. The high molecular weight HA molecules serve as delivery systems of both moisture and, especially, the small HA oligomers to the dermis where they stimulate the skin cells (keratinocytes, fibroblasts, etc.) to produce new hyaluronic acid, new collagen and elastin and other connective tissue components necessary for restoring elasticity, firmness and softness to the skin.

Thus far, the use of small HA-oligosaccharides in cosmetic formulations has been limited (Couchman et al., U.S. Pat. No. 4,761,401). In contrast, in the scientific and medical literature, several studies have already demonstrated their therapeutic potential for treatment of a variety of diseases such as cancer, asthma, etc. (Alamiz, L. et al. Glycobiology, 16, 359, 2006; Asari, A. U.S. Patent Application No. 2008012983). Moreover, it has also been shown HA oligosaccharides stimulate the synthesis of elastin (Joddar, B. and Ramamurthi, A. Biomaterials 33, Nov. 27, 5698, 2006; Joddar, B. et al. Biomaterials 27, Sep. 28, 3918, 2007).

### SUMMARY OF INVENTION

In this cosmetic formulation, the high molecular hyaluronic fractions are used as vehicles or delivery systems transporting within their matrices both water molecules and oligosaccharides to the keratinocytes and other skin cells in the dermis. Once stimulated by these oligosaccharides, the keratinocytes produce new hyaluronan, new collagen, new elastin and other connective tissue components, thereby repairing and restoring the skin to a vital and youthful state.

### DETAILED DESCRIPTION OF INVENTION

### Preparation of HA-Oligosaccharides

Purified commercial preparations of Hyaluronic acid (food grade or research grade) from bacterial sources were obtained from Lifecore or other vendors in powder form. A known amount of this material was weighed and dissolved in 0.5M sodium acetate buffer pH 5.0 containing 0.15M sodium chloride. Following the addition of testicular hyaluronidase, digestion was carried out at 37 degrees C. for 24-36 hours. A few drops of toluene were added as a bacteriostatic agent. Following digestion, the digest was dialysed against distilled water in control pore-size dialysis tubing. The dialysate containing the oligosaccharides were purified by chromatography on ion-exchange resins (Dowex-1C1) using neutral salts as eluent. The elution profile on the material was monitored by the carbazole assay of Bitter and Muir (Bitter, T. and Muir, H. M. Anal. Biochem. 4, 330, 1962). Following the removal of salt by gel filtration using either Bio-gel or Sephadex columns, the material was lyophized as a powder and its identity was established by chromatography, chemical analyses, and gel electrophoresis. This material, referred to as purified HA-oligosaccharides, was used thereafter as an active ingredient in the preparation of our cosmetic formulation, i.e. the anti-wrinkle and anti-aging moisturizing cream.

The purified HA-oligosaccharides were chemically deacetylated by using a combination of hydrazine and hydrazine sulfate, according to a published method (Dimitriev, B. A. et al., Carbohydr. Res.:29, 452-457, 1973) and used thereof. Sulfation of HA-oligosaccharides was carried out using a mixture of chlorosulfonic acid and dry pyridine according to published methods, (Balazs, E. A. et al. Acta Physiol. Scand. 23,168, 1951 and Whistler, R. L. and Speener, W. W., In Methods in Carbohydrate Chemistry (R. L. Whistler, ed.) Academic Press, NY, vol. 4, 297, 1964).

These sulfated HA-oligosaccharides were used thereafter in cosmetic formulations. For skin penetration studies, HA-oligosaccharides were coupled to fluorescein groups using isothiocyanatofluorescein according to the method of Anthony, N. et al., Carbohydr. Res. 44, 251-257, 1975.

The principal active ingredient in this invention is this hyaluronic acid (HA) aqueous mixture (0.1%) composed of equal amount of high molecular weight HA and HA-oligo-saccharides. In this mixture or composition, the high moleculuar weight HA fractions serve as delivery systems carrying both water and the HA-oligosaccharides molecules to the dermis for stimulating skin cells (keratinocytes, fibroblasts, etc.,) to produce new HA, new collagen and other connective tissue components.

The preferred experimental protocol (protocol #3) for preparing the anti-wrinkle and anti-aging moisturizer is outlined below:

To an amount of water corresponding to 67.34% of the total weight, 0.05% of high molecular weight hyaluronic acid (HA) and 0.05% HA-oligosaccharides are added. The water mixture is heated and maintained at 65 degrees C. This water-based HA mixture is emulsified by adding stearic acid (8%) and cetyl alcohol (0.75%). Sunflower oil (11%), shea butter (3%), safflower oil (0.5%) and carrot seed oil (0.2%) are added as emolients. Sodium PCA (3%) and triethanolamine (3%) are added as humectant and neutralizer, respectively. Comfrey extract (0.3%) and fennel extract (0.3%) are added as botanicals. Propylene glycol (2%) is used as solvent. Propyl paraben (0.1), methyl paraben (0.1%) and Ethylenediamine-tetraacetate, EDTA (0.05) are added as preservatives. Retinyl palmitate (Vit-A, 0.05%) and tocopheryl acetate (Vit-E, 0.1%) are added. Eucalyptus oil (0.01%) is added for its anti-bacterial and fungicidal effect. Essential oils (0.1-0.2%) are added as fragrance.

Several other protocols were generated by varying the concentrations of the various ingredients in preparing different experimental batches of moisturizer. Protocol #3 was selected as the quality of the cream prepared by this protocol was found to be maximal, i.e., best texture, consistency, stability, etc.

Similar results were also obtained when a mixture of high molecular weight hyaluronic acid and deacetylated HA-oligosaccharides was used for preparing the anti-wrinkle and anti-aging cream. In addition, batches containing deacetylated oligosaccharides revealed to be substantially beneficial to the removal of skin wrinkles and fine lines.

By varying the % of the ingredients, it is important to note that the anti-wrinkle and anti-aging cream can be made into a different product, i.e., a lotion, a serum, an under eye cream, etc. Such products would also be used in different parts of the body (hands, feet, face, etc.)

At present, a complete mechanism responsible for the repair and rejuvenation of adult human skin following the use of our anti-aging cream, remains to be firmly established as our various studies are still in progress. It is important to note that observations obtained from our preliminary studies provide unequivocal evidence of its beneficial effects. For example, a small clinical trial involving twenty five to thirty (25 to 30) women has already exceeded our expectations. By applying to the neck and face twice daily for thirty days (30), in the morning and at bed time an amount corresponding to 1 to 2 ml, they all have reported that wrinkles have lessened; skin feels softer, smoother and firmer. Note: During the 30-day treatment and after, no redness, rashes or abnormalities of any kind could be observed. It was therefore concluded that this cosmetic formulation is not irritating or immunologically active when applied to human skin and other animals. For skin penetration studies, special samples containing HA-oligosaccharides coupled to fluorescein groups were prepared. This material was applied to half the dorsal skin of six (6) nude or hairless mice. The skin was examined after 4, 8, 12 and 24 hrs., using light microscope. The results of these tests revealed complete penetration to the deeper layers of the skin. Experiments with artificial skin were carried out in dessicators at constant temperature (20 degree-25 degree C.) and constant humidity (45, 55 and 70% relative humidity).

What is claimed is:

1. A method of combining low and high molecular weight hyaluronan to treat skin conditions in humans and other mammals.

2. A method of claim 1 in which the mixture comprises equal amounts of high molecular weight hyaluronan fractions (0.5 to 2.0 million Daltons) and low molecular weight saturated hyaluronic acid oligosaccharides (500 to 12,000 daltons molecular weight).

3. A method of claim 2, wherein said mixture of hyaluronan is used to replace transepidermal water loss.

4. A method of claim 2 wherein said mixture of hyaluronan is used to prevent damage to skin elastic fibers

5. A method of claim 2 wherein said mixture of hyaluronan is used to optimize delivery of hyaluronan to the dermis

6. A method of claim 2 wherein said mixture of hyaluronan is used to produce new collagen and other extracellular matrix components

7. A method of claim 2 wherein said mixture of hyaluronan is used to stimulate the growth of new skin keratinocytes

8. A method of claim 2 wherein the said mixture of hyaluronan is used as a means of carrying both water and hyaluronan oligosaccharides to the dermis.

9. A method of claim 2 wherein the saturated HA-oligosaccharides are produced by hydrolysis of high molecular weight hyaluronic acid with testicular hyaluronidase and subsequent dialysis of the hydrolysates again distilled dionized water, using dialysis membranes of control pore sizes.

10. A method of claim 9 wherein the hyaluronan oligosaccharides are further purified by column chromatography on ion-exchange resins and subsequent elution with neutral salt solutions (potassium or sodium).

11. A method of claim 10 wherein the purified hyaluronan oligosaccharides are chemically deacetylated or sulfated for their use in cosmetic formulations.

12. A method of claim 11 wherein the deacetylated hyaluronan oligosaccharides of are mixed with equal amounts (w/w) of high molecular weight hyaluronan

13. A method of claim 10 wherein the hyaluronan oligosaccharides are coupled with isothiocyanatofluorescein to produce fluorescein oligosaccharides to determine their ability to penetrate the dermis in vitro and in vivo, using artificial skin and nude mice, respectively.

14. A method of claim 1 wherein the skin condition is one or more of the following: dry skin, wrinkled skin, acne, sunburn, etc.,

15. A method of claim 2 wherein the amount of high molecular weight hyaluronan varies from 1 microgram per ml of cream to 100 mg per ml of cream.

16. A method of claim 2 wherein the amount of low molecular weight hyaluronan oligosaccharides varies from 1 microgram per ml of cream to 100 mg per ml of cream.

17. A method of claim 2, wherein said mixture of hyaluronan is added to a cream vehicle composed of 12% oils, fatty acid, cetyl alcohol, shea butter, botanicals extracts, vitamins, essential oils as fragrance and preservatives.

18. A method of claim 17 wherein said cream contains shea butter at a concentration of 30 mg per ml of cream.
